(19) United States
(12) Patent Application Publication
(10) Pub. No.: US 2017/0287129 A1
(43) Pub. Date: Oct. 5, 2017

(54) IMAGE GENERATION APPARATUS AND IMAGE GENERATION METHOD

(71) Applicant: SEIKO EPSON CORPORATION, Tokyo (JP)

(72) Inventor: Ryoki WATANABE, Matsumoto-shi (JP)

(73) Assignee: SEIKO EPSON CORPORATION, Tokyo (JP)

(21) Appl. No.: 15/473,056

(22) Filed: Mar. 29, 2017

(30) Foreign Application Priority Data
Apr. 1, 2016 (JP) 2016-074036

(51) Int. Cl. G06T 7/00; G06T 11/60; A61B 8/14; A61B 8/08; A61B 8/00

(52) U.S. Cl. CPC G06T 7/0012; A61B 8/5207; A61B 8/461; A61B 8/14; A61B 8/467; A61B 8/4444; G06T 11/60; G06T 2207/10132

(57) ABSTRACT

An image generation apparatus includes an arithmetic processing unit that generates an ultrasonic image based on a received signal obtained by receiving a reflected wave from a subject of an ultrasonic wave incident on the subject. The arithmetic processing unit calculates an attenuation feature value at each position in an incidence direction of the ultrasonic wave based on the received signal, performs signal processing on the attenuation feature value at each position in the incidence direction, and generates an attenuation feature image using the attenuation feature value subjected to the signal processing.

### BACKGROUND

### 1. Technical Field

The present invention relates to an image generation apparatus and an image generation method of generating an ultrasonic image.

### 2. Related Art

In ultrasonic measurement apparatuses that obtain biological information regarding subjects using ultrasonic waves, there is a problem such as an acoustic shadow. Since ultrasonic waves incident on subject propagate inside the subjects while reflected from boundary surfaces of biological tissues such as muscles, blood vessels, and bones, it is possible to understand the structures of the biological tissues from received signals of reflected waves (ultrasonic echoes) of the ultrasonic waves. However, when there are strong reflection bodies, such as bones or stones, strongly reflecting ultrasonic waves, signal strengths arriving at biological tissues at the back of the strong reflection bodies may deteriorate, which cause an acoustic shadow.

As a technology for improving such an acoustic shadow, for example, there is a known technique for obtaining an acoustic shadow effect coefficient which is a value according to the degree of presence of an acoustic shadow in a region at the back of a high-luminance portion from an average luminance of the high-luminance portion and the region at the back of the high-luminance portion in a tomographic image obtained from reflected waves of ultrasonic waves and correcting luminance of the region at the back of the high-luminance portion using the coefficient (see the paragraphs [0066] to [0072] of JP-A-2005-103129).

In the technique disclosed in JP-A-2005-103129, however, it may be difficult to say that a region with low luminance in which an acoustic shadow is considered to occur is detected and luminance values of high-luminance regions in the periphery of the region are averaged so that a sufficient improvement effect of the acoustic shadow is obtained.

Incidentally, even in a case in which an acoustic shadow occurs, it is useful to ascertain where the acoustic shadow occurs since a spot of the acoustic shadow is closely observed.

### SUMMARY

An advantage of some aspects of the invention is to provide a technology for enabling a user to easily ascertain a spot related to an acoustic shadow in an ultrasonic image visually.

A first aspect of the invention is directed to an image generation apparatus including an arithmetic processing unit that generates an ultrasonic image based on a received signal obtained by receiving a reflected wave from a subject of an ultrasonic wave incident on the subject. The arithmetic processing unit performs calculation of an attenuation feature value at each position in an incidence direction of the ultrasonic wave based on the received signal, signal processing on the attenuation feature value at each position in the incidence direction, and generation of an attenuation feature image using the attenuation feature value subjected to the signal processing.

As another aspect of the invention, the aspect of the invention may be configured as an image generation method of generating an ultrasonic image based on a received signal obtained by receiving a reflected wave from a subject of an ultrasonic wave incident on the subject. The method includes: calculating an attenuation feature value at each position in an incidence direction of the ultrasonic wave based on the received signal; performing signal processing on the attenuation feature value at each position in the incidence direction; and generating an attenuation feature image using the attenuation feature value subjected to the signal processing.

According to the first aspect and the like of the invention, it is possible to calculate the attenuation feature value at each position in the incidence direction of the ultrasonic wave (incidence direction position) and generate the attenuation feature image by performing signal processing on the attenuation feature value of each incidence direction position. According to the attenuation feature image, the user can easily ascertain a spot related to the acoustic shadow in the ultrasonic image visually.

As a second aspect of the invention, the image generation apparatus according to the first aspect of the invention may be configured such that the signal processing includes normalization of the attenuation feature value.

According to the second aspect of the invention, it is possible to normalize the attenuation feature value and to image the normalized attenuation feature value.

As a third aspect of the invention, the image generation apparatus according to the first aspect of the invention may be configured such that the signal processing includes differentiation of the attenuation feature value in the incidence direction.

According to the third aspect of the invention, it is possible to differentiate the attenuation feature value in the incidence direction of the ultrasonic wave and to image the differentiated attenuation feature value.

As a fourth aspect of the invention, the image generation apparatus according to the third aspect of the invention may be configured such that the generation of the attenuation feature image includes identification display of a portion in which a predetermined abrupt drop condition indicating that the differentiated attenuation feature value is considerably lowered in the incident direction is satisfied.

According to the fourth aspect of the invention, it is possible to perform the identification display on a portion in which the attenuation feature value is considerably lowered in the incidence direction.

As a fifth aspect of the invention, the image generation apparatus according to any one of the first to fourth aspects of the invention may be configured such that the arithmetic processing unit further performs control of superimposition display or parallel display of the ultrasonic image and the attenuation feature image.

According to the fifth aspect of the invention, it is possible to display the ultrasonic image and the attenuation feature image in a superimposition manner (superimposition display) or display the ultrasonic image and the attenuation feature image in parallel (parallel display).

As a sixth aspect of the invention, the image generation apparatus according to any one of the first to fifth aspects of the invention may be configured such that the calculation of the attenuation feature value is calculation of an attenuation correction value for cancelling attenuation of the received signal using an incident signal strength of the ultrasonic wave and a reception signal strength of the reflected wave.

According to the sixth aspect of the invention, it is possible to calculate the attenuation correction value for cancelling attenuation of the received signal as the attenuation feature value.

As a seventh aspect of the invention, the image generation apparatus according to anyone of the first to fifth aspects of the invention may be configured such that calculation of the attenuation feature value is calculation of an attenuation strength value of the received signal using an incident signal strength of the ultrasonic wave and a reception signal strength of the reflected wave.

According to the seventh aspect of the invention, it is possible to calculate the attenuation strength value of the received signal as the attenuation feature value.

### BRIEF DESCRIPTION OF THE DRAWINGS

The invention will be described with reference to the accompanying drawings, wherein like numbers reference like elements.

FIG. 1 is a diagram illustrating an example of a system configuration of an image generation apparatus.

FIG. 2 is a diagram illustrating an example of an ultrasonic image.

FIG. 3 is a diagram illustrating a simple ultrasonic wave propagation model used to describe calculation of an attenuation feature value.

FIG. 4 is a diagram illustrating a graph of attenuation correction values.

FIG. 5 is a diagram illustrating an example of an acoustic shadow portion image.

FIG. 6 is a diagram illustrating a differentiation result of the attenuation correction value in FIG. 4.

FIG. 7 is a diagram illustrating an example of an acoustic shadow causing portion image.

FIG. 8 is a diagram illustrating an example of an acoustic shadow generating portion image.

FIG. 9 is a diagram illustrating another example of an acoustic shadow generating portion image.

FIG. 10 is a block diagram illustrating an example of a functional configuration example of the image generation apparatus.

FIG. 11 is a flowchart illustrating the flow of a process of generating an ultrasonic image.

### DESCRIPTION OF EXEMPLARY EMBODIMENTS

Hereinafter, preferred embodiments of the invention will be described with reference to the drawings. The embodiments to be described below do not limit the invention and modes to which the invention can be applied are not limited to the following embodiments. In the description of the drawings, the same reference numerals are given to the same portions.

### Overall Configuration

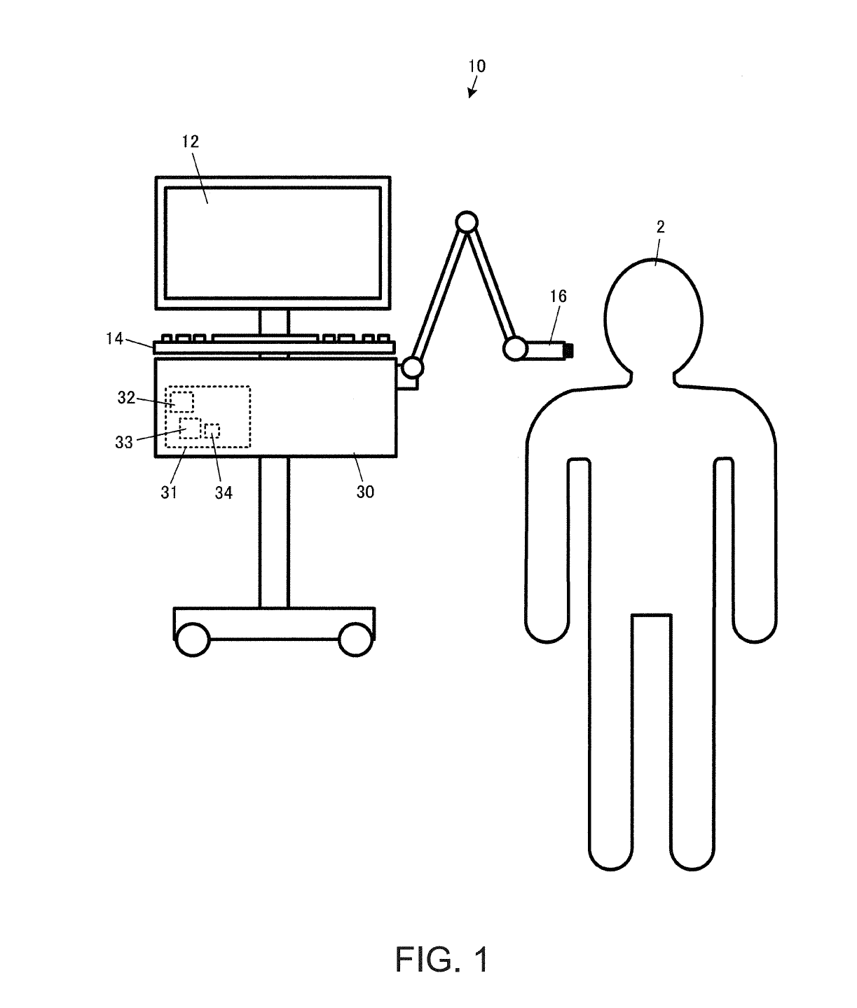

FIG. 1 is a diagram illustrating an example of a system configuration of an image generation apparatus 10 according to the embodiment. The image generation apparatus 10 includes a touch panel 12 that serves as both a unit performing image display of a measurement result or operation information and a unit performing an operation input, a keyboard 14 that performs an operation input, an ultrasonic probe (probe) 16, and a processing device 30. The image generation apparatus 10 acquires biological information regarding a subject 2 through ultrasonic measurement.

The ultrasonic probe 16 includes a plurality of arranged ultrasonic elements (ultrasonic vibrators) that transmit and receive ultrasonic waves. The ultrasonic element (hereinafter also simply referred to as an “element”) is an ultrasonic transducer that mutually converts ultrasonic waves and electric signals, transmits a pulse signal of an ultrasonic wave with a few of MHz to tens of MHz and receives the reflected wave. Before the ultrasonic measurement, the ultrasonic probe 16 is put on a part (target part) of the subject 2 according to a measurement purpose.

The processing device 30 contains a control substrate 31 and is connected to be able to transmit and receive a signal to and from each device unit of the touch panel 12, the keyboard 14, and the ultrasonic probe 16. A central processing unit (CPU) 32, a storage medium 33 such as an integrated circuit (IC) memory or a hard disk in addition to an application specific integrated circuit (ASIC), a field programmable gate array (FPGA), or any of various integrated circuits, and a communication IC 34 that realizes data communication with an external device are mounted on the control substrate 31. The processing device 30 performs a process necessary to acquire biological information in addition to ultrasonic measurement when the CPU 32 or the like executes a program stored in the storage medium 33.

Specifically, the image generation apparatus 10 causes (transmits) an ultrasonic beam to be incident on the subject 2 from the ultrasonic probe 16 under the control of the processing device 30 and receives a reflected wave (ultrasonic echo) to perform ultrasonic measurement. Then, positional information of a structure inside an organism of the subject 2 or reflected wave data changed over time is generated by performing amplification and signal processing on the received signal of the reflected wave. The ultrasonic measurement is performed repeatedly at a predetermined period. A measurement unit at the predetermined period is referred to as a “frame”.

The reflected wave data includes an image of each mode, so-called A, B, M, and Doppler modes. The A mode is a mode in which an amplitude (A mode image) of a reflected wave is displayed using a sampling point row sequence of a received signal in a scanning line direction of an ultrasonic beam (an incidence direction of ultrasonic waves) as a first axis and using a reception signal strength of the reflected wave at each sampling point as a second axis. The B mode is a mode in which a 2-dimensional ultrasonic image (B mode image) of a structure inside an organism visualized by converting an amplitude (A mode image) of a reflected wave obtained by scanning an ultrasonic beam within a predetermined probe scanning range (scanning angle) into a luminance value is displayed.

### Overview

The image generation apparatus 10 performs signal processing on the reflected wave data and performs (1) identification display of an acoustic shadow portion, (2) identification display of an acoustic shadow causing portion, and (3) identification display of an acoustic shadow generating portion in an ultrasonic image. The acoustic shadow causing portion refers to a causing portion that causes an acoustic shadow (for example, a strong reflector) and an acoustic shadow generating portion refers to a spot in which an acoustic shadow occurs due to the acoustic shadow causing portion. The acoustic shadow portion indicates an entire region including a region in which an acoustic shadow occurs and the acoustic shadow causing portion, and the acoustic shadow generating portion.

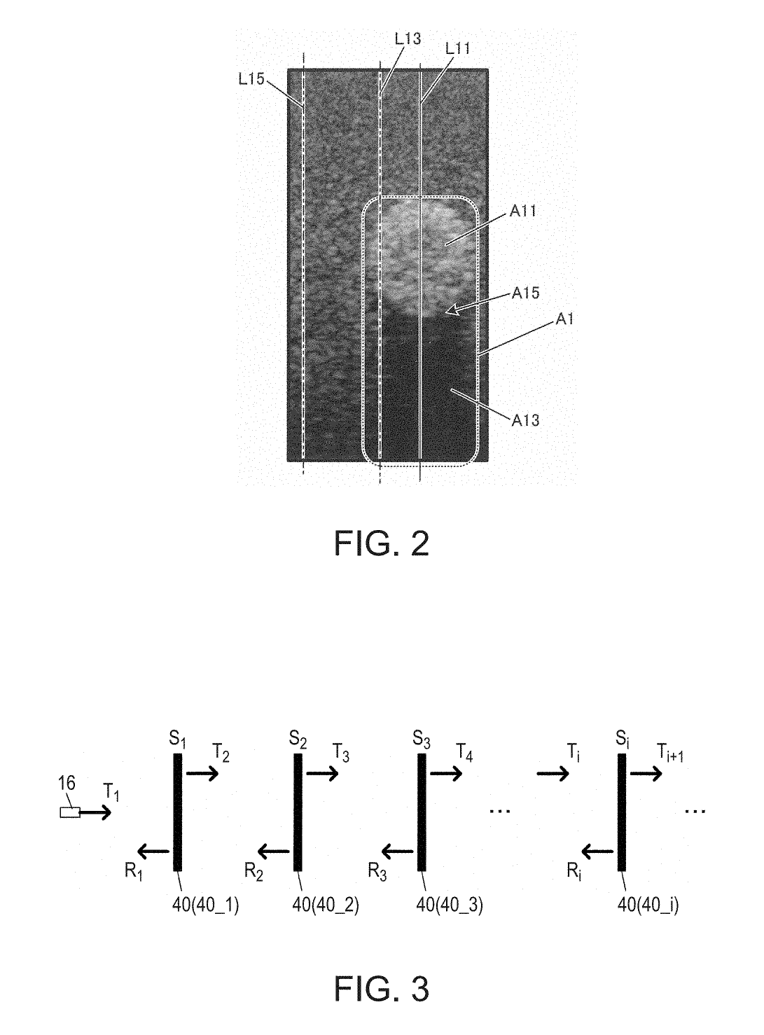

Here, an acoustic shadow is “a stripe-shaped low echo region or a non-echo region which occurs in the dorsal side of a medium from which an ultrasonic wave is strongly reflected”. FIG. 2 is a diagram illustrating an example of an ultrasonic image of a target part obtained as a B mode image. The middle and upper sides of FIG. 2 is an organism surface side (an ultrasonic incidence side) and indicates an ultrasonic image of the subject 2 including a strong reflector A11. As illustrated in FIG. 2, when there is the strong reflector A11 strongly reflecting an ultrasonic wave to a target part, it can be known that an acoustic shadow A13 that has low luminance, that is, a small reception signal strength, occurs on the dorsal surface side of the strong reflector A11 when viewed on the ultrasonic incidence side. In this example, the strong reflector A11 is an acoustic shadow causing portion, the dorsal surface portion A15 of the strong reflector A11 is an acoustic shadow generating portion, and a region A1 including the strong reflector A11 and the acoustic shadow A13 is an acoustic shadow portion. Principle

An ultrasonic wave incident on the subject 2 propagates while being attenuated inside the subject 2. As occurring attenuation, there are mainly three types of attenuations, spread attenuation, absorption attenuation, and diffusion attenuation. The spread attenuation is attenuation caused since a sound wave spreads in a spherical shape. The absorption attenuation is attenuation caused when an acoustic energy is absorbed into a medium and is subjected to heat conversion. The diffusion attenuation is attenuation caused since a medium is irregular. The diffusion attenuation is considered to be a main cause of the acoustic shadow. Accordingly, a case in which a medium A propagating an ultrasonic wave includes a different medium B therein will be considered focusing on the diffusion attenuation. Here, it is assumed that there is no spread attenuation and absorption attenuation of an ultrasonic wave.

An acoustic impedance Z₁ of the medium A is obtained by a product of an average density ρ₁ and an average sound speed c₁ of the medium A and an acoustic impedance Z₂ of the medium B is obtained by a product of an average density ρ₂ and an average sound speed c₂ of the medium B (Equation (1) below).

Z₁=ρ₁×c₁

Z₂=ρ₂×c₂ (1)

When an ultrasonic wave propagating the medium A is reflected in a boundary surface of the media A and B, a reflection ratio S is expressed in Equation (2) below using the acoustic impedances Z₁ and Z₂ of the media A and B.

$$S = {( \frac{{Z}_{2} - {Z}_{1}}{{Z}_{2} + {Z}_{1}} )}^{2} \qquad ( 2 )$$

Then, transmittance T of an ultrasonic wave transmitting the boundary surface of the media A and B is expressed in Equation (3) below.

$$T = 1 - S = \frac{4 {Z}_{1} {Z}_{2}}{{( {Z}_{2} + {Z}_{1} )}^{2}} \qquad ( 3 )$$

From Equation (3), it can be understood that as a difference between the acoustic impedance Z₁ of the medium A and the acoustic impedance Z₂ of the medium B is larger, the reflection ratio S of an ultrasonic wave in the boundary surface of the media A and B is larger and the transmittance T is smaller. Accordingly, in the boundary surface of the different media A and B, the signal strength of an ultrasonic wave transmitting the boundary surface decreases as the reflection ratio S is larger (as the transmittance ratio T is smaller), and thus an attenuated signal is generated. Thus, the acoustic shadow A13 illustrated in FIG. 2 occurs. In the embodiment, the degree of attenuation is quantified and used to specify an acoustic shadow portion, an acoustic shadow causing portion, and an acoustic shadow generating portion in an ultrasonic image.

The degree of attenuation is quantified by obtaining an attenuation correction value which is one of the attenuation feature values. FIG. 3 is a diagram illustrating a simple ultrasonic wave propagation model used to describe calculation of an attenuation correction value. FIG. 3 illustrates a case in which an ultrasonic wave with an incidence signal strength T₁ is incident on a subject that has a plurality of medium boundary surfaces 40 from the ultrasonic probe 16 to the right side of FIG. 3. Here, it is assumed that there is no spread attenuation and absorption attenuation of an ultrasonic wave.

In the subject illustrated in FIG. 3, there are a plurality of medium boundary surfaces 40_i (where i=1, 2, . . . ) which are present to face each other in the ultrasonic incidence direction (a depth direction from a biological surface in the embodiment). Thus, an ultrasonic wave incident from the ultrasonic probe 16 propagates while being reflected from or transmitted through the medium boundary surfaces 40. A reflection ratio Sᵢ of an i-th medium boundary surface 40_i is decided by the acoustic impedance Z of two media which are boundaries in Equation (2). Then, a reception signal strength (reflection strength) Rᵢ of a reflected wave of the ultrasonic wave from the i-th medium boundary surface 40_i is obtained by a product of an incident signal strength (incidence strength) Tᵢ of the ultrasonic wave incident on the medium boundary surface 40_i and the reflection ratio Sᵢ by the medium boundary surface 40_i (Equation (4) below).

Rᵢ=Tᵢ×Sᵢ (4)

Specifically, the incidence strength T₁ on the first medium boundary surface 40_1 is an incident signal strength T₁ of an ultrasonic wave from the ultrasonic probe 16. The incident strength Tᵢ on a second or subsequent medium boundary surface 40_i (where i=2, 3, . . . ) is a transmission strength of an ultrasonic wave of a front (i−1)-th medium boundary surface 40_(i−1) and is obtained by a difference between the incidence strength Tᵢ₋₁ on the medium boundary surface 40_(i−1) and a reflection strength Rᵢ₋₁ from the medium boundary surface 40_(i−1) (Equation (5) below).

Tᵢ=Tᵢ₋₁−Rᵢ₋₁ (5)

That is, the incidence strength Tᵢ of each medium boundary surface 40_i (where i=1, 2, . . . ) can be expressed in Equation (6) below.

$$\begin{matrix}\begin{matrix}{T}_{2} = {T}_{1} - {R}_{1} \\ {T}_{3} = {T}_{2} - {R}_{2} = ( {T}_{1} - {R}_{1} ) - {R}_{2} \\ {T}_{4} = {T}_{3} - {R}_{3} = ( {T}_{1} - {R}_{1} - {R}_{2} ) - {R}_{3} \\ ⋮ \\ {T}_{i} = {T}_{1} - {\sum }_{j = 1}^{i - 1} {T}_{j}\end{matrix} & ( 6 )\end{matrix}$$

The reflection strength Rᵢ from the medium boundary surface 40_i becomes a reception signal strength in the ultrasonic probe 16. At this time, a received signal of the reflected wave from the i-th medium boundary surface 40_i becomes an attenuated signal in such a manner that a part of the ultrasonic wave is reflected by a front (i−1)-th medium boundary surface 40_j (where j=1, 2, . . . and i−1) and the incidence strength Tᵢ is lowered.

Then, when there is no front medium boundary surface 40_j (where j=1, 2, . . . and i−1) previous to the i-th medium boundary surface 40_i, that is, an ideal state in which an ultrasonic wave with the incidence strength T₁ is incident on an i-th medium boundary surface 40_i from the ultrasonic probe 16 is considered, the reflection strength Rᵢ from the medium boundary surface 40_i is expressed in Equation (7) below.

Rᵢ=T₁×Sᵢ (7)

However, an actual reflection strength Rᵢ from the i-th medium boundary surface 40_i is less than the reflection strength Rᵢ in the ideal state due to diffusion attenuation, as expressed in Equation (4) above. Accordingly, as indicated in Equation (8) below, the actual reflection strength Rᵢ is multiplied by a predetermined attenuation correction value αᵢ to be identical to the reflection strength Rᵢ in the ideal state.

T₁×Rᵢ=αᵢ×(Tᵢ×Rᵢ) (8)

From Equation (8), the attenuation correction value αᵢ of the i-th medium boundary surface 40_i is expressed in Equation (9) below.

$${α}_{i} = \frac{{T}_{i}}{{T}_{i} - {\sum }_{j = 1}^{i - 1} {R}_{j}} \qquad ( 9 )$$

When the attenuation correction value αᵢ obtained in this way is multiplied by the actual reflection strength Rᵢ, attenuation of the received signal on the corresponding medium boundary surface 40_i is cancelled. Accordingly, the attenuation correction value αᵢ indicates the degree of a decrease of the actual reflection strength (a reception signal strength) Rᵢ compared to the reflection strength Rᵢ in the ideal state on the medium boundary surface 40_i, that is, the degree of attenuation.

### (1) Identification Display of Acoustic Shadow Portion

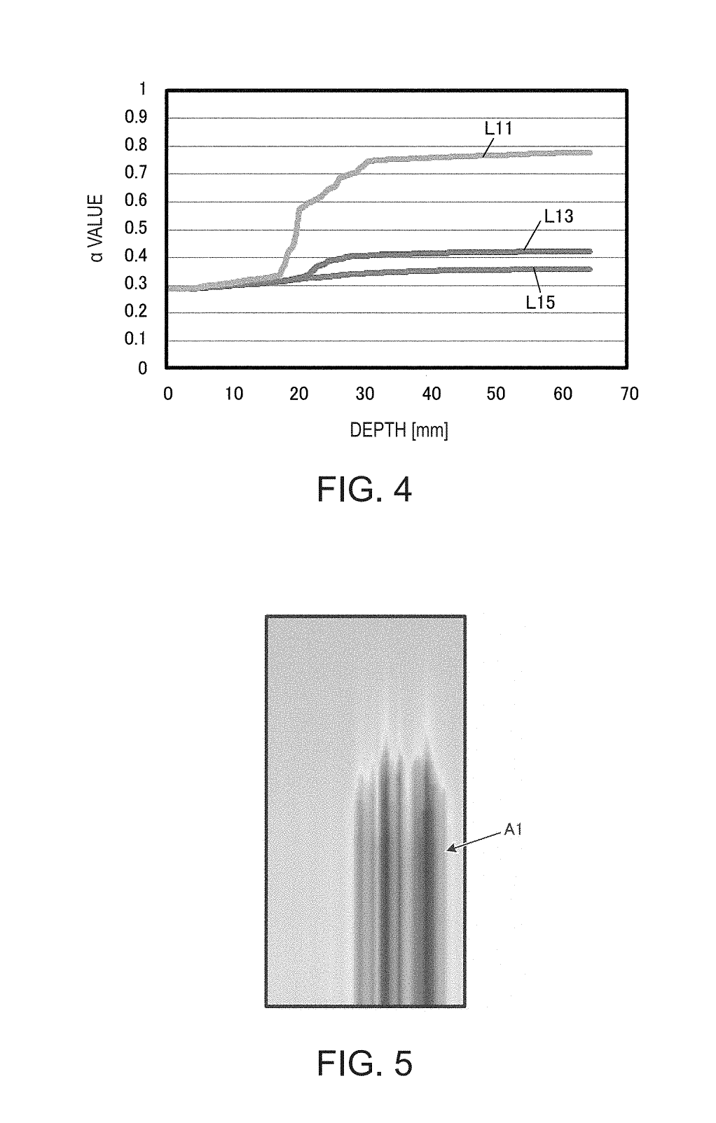

FIG. 4 is a diagram illustrating a graph of attenuation correction values αᵢ related to three scanning lines L11, L13, and L15 of interest calculated from A mode images related to the scanning lines L11, L13, and L15 of interest in FIG. 2 on the same axis. The normalized attenuation correction values αᵢ are illustrated. The attenuation correction value αᵢ can be obtained from Equation (9) using each sampling point as i. In FIG. 4, the horizontal axis represents a distance of each sampling point from an incident position (biological surface position) of an ultrasonic wave. Of the scanning lines L11, L13, and L15 of interest, two scanning lines L11 and L13 of interest are scanning lines passing through the strong reflector A11. FIG. 5 is a diagram illustrating an example of an acoustic shadow portion image which is one of the attenuation feature images. The acoustic shadow portion image can be obtained by normalizing the attenuation correction values αᵢ related to all the scanning lines and imaging the normalized attenuation correction values αᵢ (converting the normalized attenuation correction values αᵢ into a luminance value).

As illustrated in FIG. 4, the attenuation correction values αᵢ related to the scanning lines L11 and L13 of interest passing through the strong reflector A11 considerably increase at an incidence direction position of a front surface (a surface on the incidence side of an ultrasonic wave) of the strong reflector A11. In contrast, the attenuation correction value αᵢ related to the scanning line L15 of interest not passing through the strong reflector A11 gently increases without involving an abrupt change.

Here, as described above, the dorsal surface portion A15 of the strong reflector A11 is an acoustic shadow generating portion and the acoustic shadow A13 is a region on the dorsal surface side of the strong reflector A11. Therefore, the attenuation correction values αᵢ are large in the entire region of the acoustic shadow portion A1 in an ultrasonic image and are small in a region other than the acoustic shadow portion A1. Accordingly, by imaging the attenuation correction values αᵢ, as illustrated in FIG. 5, it is possible to perform identification display of the acoustic shadow portion A1 in the ultrasonic image. Accordingly, when the user views the acoustic shadow portion image, the user can easily ascertain a spot related to the acoustic shadow in the ultrasonic image, in particular, the acoustic shadow portion A1.

### (2) Identification Display of Acoustic Shadow Causing Portion

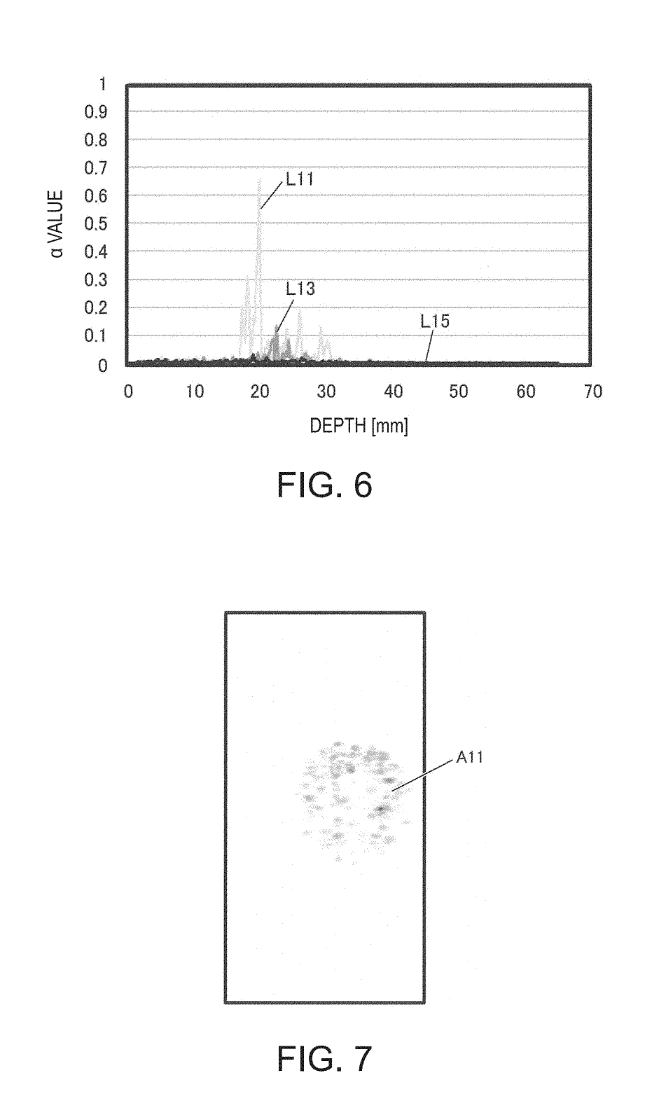

FIG. 6 is a diagram illustrating a graph of a differentiation result obtained by differentiating the attenuation correction values αᵢ related to the scanning lines L11, L13, and L15 of interest illustrated in FIG. 4 in the incidence direction (that is, the direction of the scanning lines) and illustrating derivative values of the normalized attenuation correction values αᵢ on the same axis. FIG. 7 is a diagram illustrating an example of an acoustic shadow causing portion image which is one of the attenuation feature images. The acoustic shadow causing portion image can be obtained by differentiating the attenuation correction values αᵢ related to all the scanning lines in the incidence direction, normalizing the derivative values, and imaging the normalized derivative values.

As illustrated in FIG. 6, when the attenuation correction values αᵢ related to the scanning lines L11 (light gray line) and L13 (dark gray line) of interest passing through the strong reflector A11 are differentiated in the incidence direction, a plurality of peaks are shown in the incidence direction positions of the strong reflector A11. In contrast, in the scanning line L15 (black line) of interest not passing through the strong reflector A11, a derivative value of the attenuation correction value αᵢ is not considerably changed and remains to be a small value. Accordingly, by imaging a derivative value of the attenuation correction value a, as illustrated in FIG. 7, it is possible to perform identification display of the strong reflector (the acoustic shadow causing portion) A11 in the ultrasonic image. Accordingly, when the user views the acoustic shadow causing portion image, the user can easily ascertain a spot related to the acoustic shadow in the ultrasonic image, in particular, the acoustic shadow causing portion A11.

### (3) Identification Display of Acoustic Shadow Generating Portion

Since the acoustic shadow generating portion is the dorsal surface portion A15 of the strong reflector A11, a location in which the derivative value of the attenuation correction value αᵢ is considerably low is considered to be an incidence direction position of the acoustic shadow generating portion. In the embodiment, for example, “the fact that a derivative value of a sampling point on the back side in the incidence direction is equal to or less than 1/10 of a derivative value of a sampling point on the front side in the incidence direction”, compared to the derivative value of the attenuation correction value αᵢ between adjacent sampling points, is determined as an abrupt drop condition. Then, an incidence direction position of the sampling point which satisfies the abrupt drop condition is specified as an acoustic shadow generating portion, and an acoustic shadow generating portion image which is one of the attenuation feature images in which the acoustic shadow generating portion is subjected to the identification display is generated.

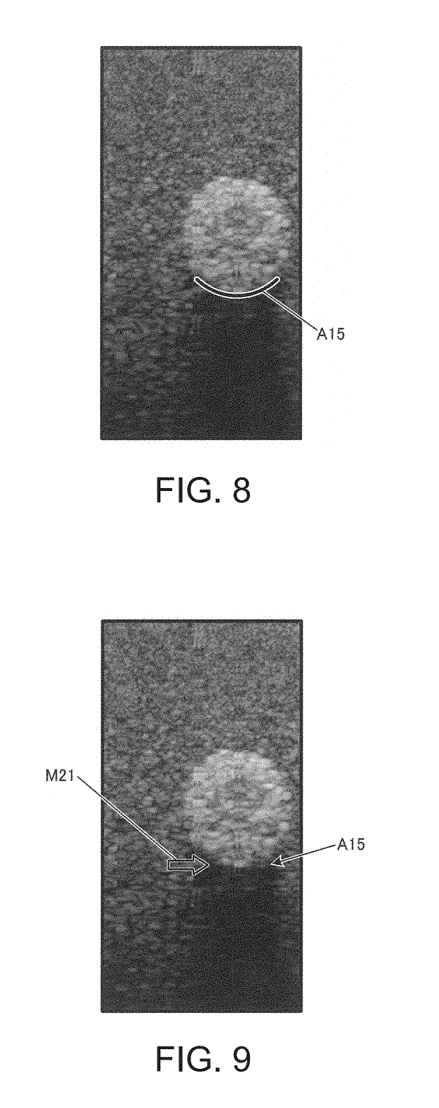

FIG. 8 is a diagram illustrating an example of an acoustic shadow generating portion image. FIG. 9 is a diagram illustrating another example of an acoustic shadow generating portion image. The acoustic shadow generating portion image is an image obtained by displaying the dorsal surface portion (acoustic shadow generating portion) A15 of the strong reflector A11 in the ultrasonic image in FIG. 2 with a predetermined display color, for example, as illustrated in FIG. 8, or obtained by, for example, disposing a generating portion indication marker M2 near the acoustic shadow generating portion A15 and performing identification display on the acoustic shadow generating portion A15 in the ultrasonic image, as illustrated in FIG. 9. A region of the acoustic shadow A13 is dark. Therefore, in a case in which there is an abnormal portion such as an alveolus or a stone in the region of the acoustic shadow A13, there is a problem of the abnormal portion being easily overlooked. Therefore, when the user views the acoustic shadow generating portion image in which the acoustic shadow generating portion A15 is emphasized and displayed, the user can easily ascertain a spot related to the acoustic shadow in an ultrasonic image, in particular, the acoustic shadow generating portion A15 and can closely observe the region of the dark acoustic shadow A13 on the dorsal surface side using the identification display of the acoustic shadow generating portion A15 as a clue. Thus, it is possible to prevent the abnormal portion from being overlooked.

The identification display of each of the (1) acoustic shadow portion, (2) the acoustic shadow causing portion, and (3) the acoustic shadow generating portion described above can be performed by switching each display form of display of the acoustic shadow portion, display of the acoustic shadow causing portion, and display of the acoustic shadow generating portion. A change in the display form can be realized through a manipulation of pressing a selection button used to select each display form. The selection button may be realized as a physically disposed button switch or a software key switch formed using the touch panel 12.

When the display of the acoustic shadow portion is selected, the acoustic shadow portion image is superimposed to be displayed on the ultrasonic image. When the display of the acoustic shadow causing portion is selected, the acoustic shadow causing portion image is superimposed to be displayed on the ultrasonic image. When the display of the acoustic shadow generating portion is selected, the acoustic shadow generating portion image is displayed. For the display of the acoustic shadow portion or the display of the acoustic shadow causing portion, the acoustic shadow portion image or the acoustic shadow generating portion image may be displayed in parallel to the ultrasonic image. When the user compares the images, the user can easily ascertain a spot related to the acoustic shadow in the ultrasonic image visually, such as the acoustic shadow portion or the acoustic shadow causing portion present in the ultrasonic image.

### Functional Configuration

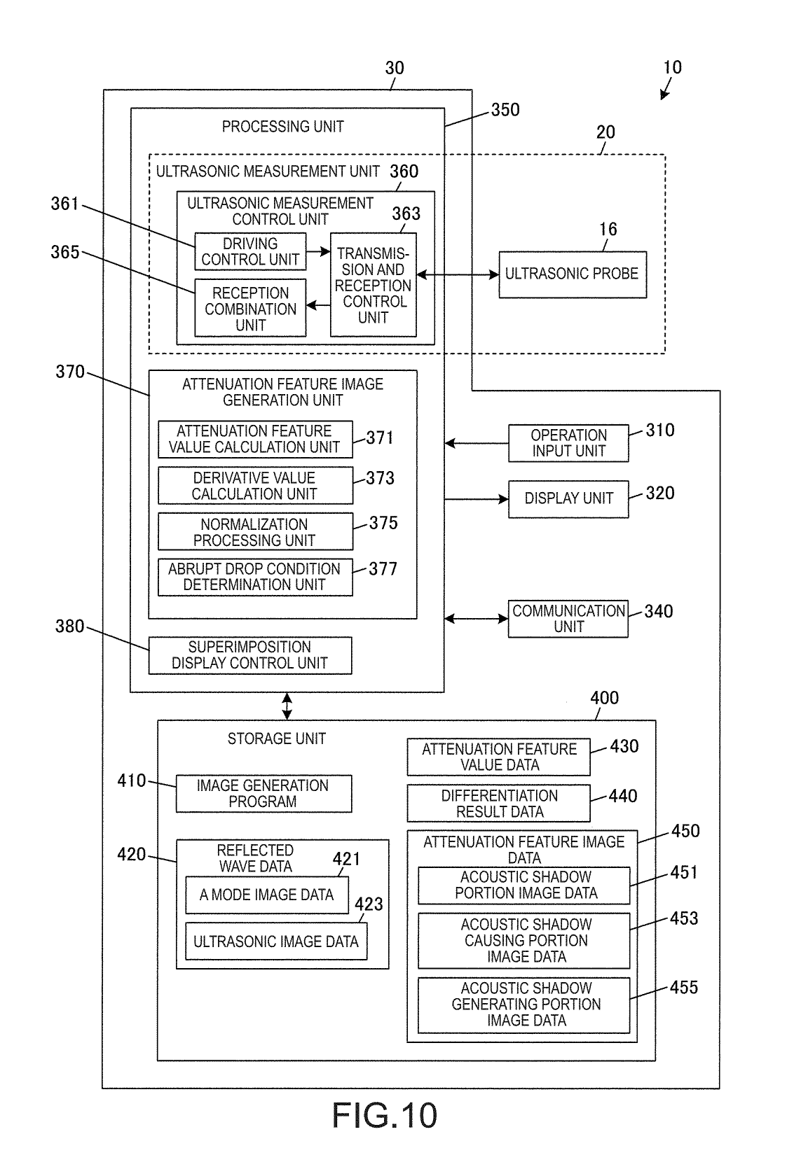

FIG. 10 is a block diagram illustrating an example of a functional configuration example of the image generation apparatus 10. The image generation apparatus 10 includes the processing device 30 and the ultrasonic probe 16. The processing device 30 includes an operation input unit 310, a display unit 320, a communication unit 340, a processing unit 350 serving as an arithmetic processing unit, and a storage unit 400.

The ultrasonic probe 16 includes a plurality of ultrasonic elements and transmits an ultrasonic wave with pulse voltage output from the processing device 30 (an ultrasonic measurement control unit 360 of the processing unit 350). Then, a reflected wave of the transmitted ultrasonic wave is received and a received signal is output to the ultrasonic measurement control unit 360.

The operation input unit 310 receives various operation inputs by the user and outputs operation input signals according to the operation inputs to the processing unit 350. The operation input unit 310 can be realized with a button switch, a lever switch, a dial switch, a track pad, a mouse, or the like. In FIG. 1, the touch panel 12 or the keyboard 14 is equivalent to the operation input unit 310.

The display unit 320 is realized by a display device such as a liquid crystal display (LCD) and performs various kinds of display based on display signals from the processing unit 350. In FIG. 1, the touch panel 12 is equivalent to the display unit 320.

The communication unit 340 is a communication device that transmits and receives data to and from the outside under the control of the processing unit 350. As a communication scheme of the communication unit 340, any of various schemes such as a form of wired connection via a cable conforming to a predetermined communication standard, a form of connection via an intermediate device also used as a charger called a cradle or the like, and a form of wireless connection using wireless communication can be applied. In FIG. 1, the communication IC 34 is equivalent to the communication unit 340.

The processing unit 350 is realized by, for example, an electronic component such as a microprocessor such as a CPU or a graphics processing unit (GPU), an ASIC, or an IC memory. The processing unit 350 performs input and output control of data with each functional unit, and performs various arithmetic processes based on a predetermined program or data, an operation input signal from the operation input unit 310, and a received signal of each element from the ultrasonic probe 16 to acquire biological information regarding the subject 2. In FIG. 1, the CPU 32 is equivalent to the processing unit 350. Each unit included in the processing unit 350 may be configured by hardware such as a dedicated module circuit.

The processing unit 350 includes an ultrasonic measurement control unit 360, an attenuation feature image generation unit 370, and a superimposition display control unit 380.

The ultrasonic measurement control unit 360 is included in the ultrasonic measurement unit 20 along with the ultrasonic probe 16. The ultrasonic measurement unit 20 performs ultrasonic measurement. The ultrasonic measurement control unit 360 can be realized in accordance with a known technology. For example, the ultrasonic measurement control unit 360 includes a driving control unit 361, a transmission and reception control unit 363, and a reception combination unit 365 and integrally controls the ultrasonic measurement.

The driving control unit 361 controls a transmission timing of an ultrasonic pulse from the ultrasonic probe 16 and outputs a transmission control signal to the transmission and reception control unit 363.

The transmission and reception control unit 363 generates a pulse voltage according to the transmission control signal from the driving control unit 361 and outputs the pulse voltage to the ultrasonic sensor 4. At this time, the transmission and reception control unit 363 performs a transmission delaying process and adjusts an output timing of a pulse voltage to each element. The transmission and reception control unit 363 performs amplification or a filtering process on a received signal input from the ultrasonic sensor 4 and outputs a process result to the reception combination unit 365.

The reception combination unit 365 performs a delaying process or the like as necessary and performs a process related to focus of the so-called received signal to generate reflected wave data.

The attenuation feature image generation unit 370 generates an attenuation feature image of each of the acoustic shadow portion image, the acoustic shadow causing portion image, and the acoustic shadow generating portion image based on a result of the ultrasonic measurement by the ultrasonic measurement unit 20. The attenuation feature image generation unit 370 includes an attenuation feature value calculation unit 371, a derivative value calculation unit 373, a normalization processing unit 375, and an abrupt drop condition determination unit 377.

The attenuation feature value calculation unit 371 calculates the attenuation correction value αᵢ of each sampling point using the incident signal strength T₁ of the ultrasonic wave from the ultrasonic probe 16 and the received signal strength of each sampling point for each scanning line.

The derivative value calculation unit 373 differentiates the attenuation correction value αᵢ of each sampling point obtained for each scanning line by the attenuation feature value calculation unit 371 in the incidence direction (that is, the direction of the scanning line).

The normalization processing unit 375 performs a process of normalizing the attenuation correction value αᵢ of each line and a process of normalizing the derivative value of the attenuation correction value αᵢ for each line.

The abrupt drop condition determination unit 377 sequentially searches for the sampling points satisfying the abrupt drop condition from the incidence side using the derivative value of the attenuation correction value αᵢ for each scanning line and specifies the incidence direction position of the acoustic shadow generating portion.

The superimposition display control unit 380 controls superimposition display or parallel display of the ultrasonic image and the acoustic shadow portion image in response to a user's operation of switching the display form or controls superimposition display or parallel display of the ultrasonic image and the acoustic shadow causing portion image.

The storage unit 400 is realized by a storage medium such as an IC memory, a hard disk, or an optical disc. The storage unit 400 stores a program for realizing various functions of the image generation apparatus 10 by operating the image generation apparatus 10 or data to be used during execution of the program in advance or temporarily stores the program or the data at the time of each process. In FIG. 1, the storage medium 33 mounted on the control substrate 31 is equivalent to the storage unit 400. The connection of the processing unit 350 and the storage unit 400 is not limited to connection by an internal bus circuit in the apparatus and may be realized by a communication line such as a local area network (LAN) or the Internet. In this case, the storage unit 400 may be realized by another external storage device other than the image generation apparatus 10.

The storage unit 400 stores an image generation program 410, reflected wave data 420, attenuation feature value data 430, differentiation result data 440, and attenuation feature image data 450.

The processing unit 350 realizes the function of the ultrasonic measurement control unit 360 or the attenuation feature image generation unit 370 by reading and executing the image generation program 410. In a case in which the functional unit is realized by hardware such as an electronic circuit, a part of the program realizing the function can be omitted.

As the reflected wave data 420, reflected wave data obtained through ultrasonic measurement repeated for each frame is stored. The reflected wave data 420 includes A mode image data 421 which is a received signal strength of each sampling point of each scanning line acquired for each frame and ultrasonic image data 423 of each frame which is a B mode image.

As the attenuation feature value data 430, the attenuation correction value αᵢ calculated by the attenuation feature value calculation unit 371 is stored for each sampling point of each scanning line. As the differentiation result data 440, the derivative value of the attenuation correction value αᵢ calculated by the derivative value calculation unit 373 is stored for each sampling point of each scanning line.

As the attenuation feature image data 450, acoustic shadow portion image data 451, acoustic shadow causing portion image data 453, and acoustic shadow generating portion image data 455 are stored as image data of the attenuation feature image.

### Flow of Process

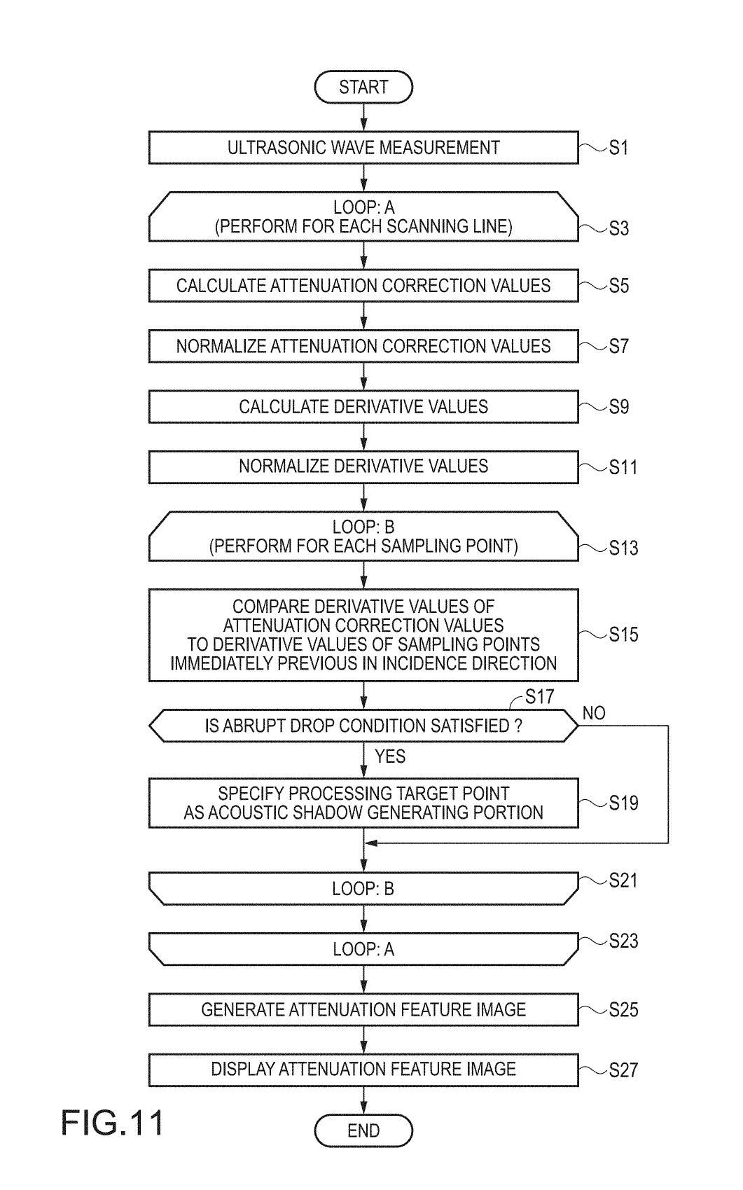

FIG. 11 is a flowchart illustrating the flow of a process of generating an attenuation feature image according to the embodiment. The process to be described here can be realized when the processing unit 350 reads the image generation program 410 from the storage unit 400 and executes the image generation program 410 to operate each unit of the image generation apparatus 10. Before the measurement, the user faces the ultrasonic probe 16 toward the body surface of the subject 2.

First, the ultrasonic measurement unit 20 performs the ultrasonic measurement to generate the reflected wave data 420 (step S1).

Subsequently, all the scanning lines are sequentially set as processing target lines and a process of a loop A is repeated (steps S3 to S23). That is, in the loop A, the attenuation feature value calculation unit 371 first sequentially calculates the attenuation correction values αᵢ for all the sampling points from the sampling points of the ultrasonic incidence side (step S5). Specifically, in accordance with Equation (9), the attenuation correction values αᵢ of the target sampling points are calculated from the incident signal strength T₁ of the ultrasonic wave from the ultrasonic probe 16 and the reflection strength (received signal strength) Rᵢ up to the sampling points on the front side in the incidence direction. Thereafter, the normalization processing unit 375 normalizes the attenuation correction value αᵢ of each sampling point (step S7).

Then, the derivative value calculation unit 373 differentiates the attenuation correction values αᵢ of the processing target lines obtained in step S5 (step S9). Thereafter, the normalization processing unit 375 normalizes the derivative value of the attenuation correction value αᵢ of each sampling point (step S11).

Subsequently, the sampling points of the processing target lines are sequentially set as the processing target points and a process of a loop B is repeated (steps S13 to S21). That is, in the loop B, the abrupt drop condition determination unit 377 first compares the derivative values of the attenuation correction values αᵢ of the processing target points to the derivative values of the attenuation correction value αᵢ of the sampling points immediately previous the processing target points in the incidence direction (step S15). Then, in a case in which the derivative values of the processing target point are equal to or less than 1/10 of the immediately previous derivative value, the abrupt drop condition determination unit 377 determines that the abrupt drop condition is satisfied (Yes in step S17) and specifies the processing target point as the acoustic shadow generating portion (step S19).

When the process of the loop B is performed on all the sampling points of the processing target lines, the process of the loop A on the processing target lines ends. Then, when the process of the loop A is performed on all the scanning lines, the attenuation feature image generation unit 370 generates the attenuation feature image (step S25). Specifically, the attenuation correction values αᵢ after the normalization in step S7 are imaged to generate the acoustic shadow portion image, the derivative values after the normalization in step S11 are imaged to generate the acoustic shadow causing portion image, and the acoustic shadow generating portion image in which the acoustic shadow generating portion specified for each sampling point in step S19 is subjected to the identification display on the ultrasonic image is generated. Thereafter, the processing unit 350 performs control such that the attenuation feature image is displayed on the display unit 320 with reference to the attenuation feature image data 450 in response to a user's operation input of giving an instruction of the display form of the attenuation feature image (step S27). At this time, in a case in which the display of the acoustic shadow portion is selected, the superimposition display control unit 380 performs control such that the superimposition display or the parallel display of the ultrasonic image and the acoustic shadow portion image is performed. In a case in which the display of the acoustic shadow causing portion is selected, the superimposition display control unit 380 performs control such that the superimposition display or the parallel display of the ultrasonic image and the acoustic shadow causing portion image is performed.

As described above, according to the embodiment, the acoustic shadow portion in the ultrasonic image can be subjected to the identification display, the acoustic shadow causing portion in the ultrasonic image can be subjected to the identification display, or the acoustic shadow generating portion in the ultrasonic image can be subjected to the identification display. Accordingly, the user can easily ascertain the spot related to the acoustic shadow such as the acoustic shadow portion, the acoustic shadow causing portion, or the acoustic shadow generating portion in the ultrasonic image visually.

In the foregoing embodiment, the attenuation correction value αᵢ is calculated as the attenuation feature value. On the other hand, an attenuation correction value βᵢ which is another attenuation feature value may be calculated and the attenuation correction value βᵢ may be used instead of the attenuation correction value αᵢ. The attenuation correction value βᵢ is expressed in Equation (10) below and can be calculated from the incident signal strength T₁ and the attenuation correction value αᵢ of the ultrasonic wave from the ultrasonic probe 16.

βᵢ=(T₁−αᵢ)−(Tᵢ−αᵢ) (10)

The entire disclosure of Japanese Patent Application No. 2016-074036 filed Apr. 1, 2016 is expressly incorporated by reference herein.

What is claimed is:

1. An image generation apparatus comprising:
an arithmetic processing unit that generates an ultrasonic image based on a received signal obtained by receiving a reflected wave from a subject of an ultrasonic wave incident on the subject,
wherein the arithmetic processing unit performs
calculation of an attenuation feature value at each position in an incidence direction of the ultrasonic wave based on the received signal,
signal processing on the attenuation feature value at each position in the incidence direction, and
generation of an attenuation feature image using the attenuation feature value subjected to the signal processing.

2. The image generation apparatus according to claim 1,
wherein the signal processing includes normalization of the attenuation feature value.

3. The image generation apparatus according to claim 1,
wherein the signal processing includes differentiation of the attenuation feature value in the incidence direction.

4. The image generation apparatus according to claim 3,
wherein the generation of the attenuation feature image includes identification display of a portion in which a predetermined abrupt drop condition indicating that the differentiated attenuation feature value is considerably dropped in the incident direction is satisfied.

5. The image generation apparatus according to claim 1,
wherein the arithmetic processing unit further performs control of superimposition display or parallel display of the ultrasonic image and the attenuation feature image.

6. The image generation apparatus according to claim 1,
wherein the calculation of the attenuation feature value is calculation of an attenuation correction value for cancelling attenuation of the received signal using an incident signal strength of the ultrasonic wave and a reception signal strength of the reflected wave.

7. The image generation apparatus according to claim 1,
wherein the calculation of the attenuation feature value is calculation of an attenuation strength value of the received signal using an incident signal strength of the ultrasonic wave and a reception signal strength of the reflected wave.

8. An image generation method of generating an ultrasonic image based on a received signal obtained by receiving a reflected wave from a subject of an ultrasonic wave incident on the subject, the method comprising:
calculating an attenuation feature value at each position in an incidence direction of the ultrasonic wave based on the received signal;
performing signal processing on the attenuation feature value at each position in the incidence direction; and
generating an attenuation feature image using the attenuation feature value subjected to the signal processing.
